(19) United States
(12) Patent Application Publication
(10) Pub. No.: US 2004/0236375 A1
(43) Pub. Date: Nov. 25, 2004

(54) Wearable, portable sonic applicator for inducing the release of bioactive compounds from internal organs

(76) Inventor: Bruce K. Redding JR., Broomall, PA (US)

Correspondence Address: Bruce K. Redding, Jr., P.O. Box 759, Broomall, PA, 19008, US

(21) Appl. No.: 10/314,378

(22) Filed: Dec. 9, 2002

Related U.S. Application Data
(60) Provisional application No. 60/348,306, filed on Jan. 15, 2002.

(51) Int. Cl. A61N 1/30

(52) U.S. Cl. 607/3

(57) ABSTRACT

The invention is a portable device for transmitting acoustical energy from an ultrasonic generator, through the exterior surface of the skin of a patient, for the purpose of increasing the release of biofunctional compounds from a targeted organ for treatment of disease or injury. In particular the invention is a means to cause the release of insulin from the patient's pancreas, to enhance the treatment of diabetes. The portable device is programmable as to acoustic frequency, intensity, and time thereby regulating the amount of insulin, which can be induced to be released in response to the sonic transmission from the patient's pancreas. The device can be programmed for steady sonic transmission or for pulsed transmissions as needed. The device is primarily aimed at Type-2 diabetics, who produce insulin internally but not necessarily in amounts or at times needed for effective biofunctionality. In such instance the invention, through the use of sonic transmissions, may be used to artificially induce the proper release of insulin from a diabetic pancreas, with or without the use of accompanying medication.

### BACKGROUND OF THE INVENTION

1. Field of the Invention

The present invention relates to a portable inducement system for enhancing insulin from the pancreas of a diabetic patient by inducing the pancreas to release greater quantities of insulin upon reaction to a sonic transmission administered through the skin via a device, which is affixed to the exterior of the body. In particular, acoustical energy delivered by a portable, self-powered, programmable ultrasonic transducer placed over the area of the patient's pancreas causes the diabetic pancreas to release insulin in response to the activation of the ultrasonic transmission.

2. Background of the Invention

The present invention relates to a portable programmable ultrasonic device, which is worn by the patient, over the area of the pancreas for the purpose of inducing the pancreas to release insulin on demand in response to the sonic transmission. Further, the portable sonic applicator may be programmed to apply acoustical energy at different times and thereby cause the release of a varying quantity of insulin over time, with or without the use of an accompanying treatment of other medicinal compounds. The portable sonic applicator may be programmed to deliver an ultrasonic transmission which will induce the pancreas of the diabetic patient to release insulin continuously (sustained release) or intermittently (pulsed release) whichever may be deemed more appropriate to the insulin management program and treatment regimen for a particular diabetic patient.

In the prior art, several medicinal compounds have been found to be suitable for Type-Two diabetes treatment as an Insulin enhancer. Essentially these drugs are used to enhance the ability of the Type-2 diabetic to more fully utilize the insulin produced by their own body. Some of the most critically needed medications are presently administered either by injection or oral dosage forms.

Recent work conducted upon cadavers using ultrasound (Langer, Edwards, Kost-MIT-1995) suggests that ultrasound applied to transdermal delivery devices and patches can enhance the penetration and/or absorption of certain low molecular weight pharmaceutical medications through the skin layer where normally low skin penetration would be expected without the use of the ultrasonic device. It has been shown that sonophoresis, the use of ultrasonic energy to enhance bioabsorption of large protein molecules or large molecule medications through the skin's outer layer, is possible when low frequencies are applied to a transdermal patch for particular medications. U.S. Pat. No. 5,947,921 to Kost et al. describes a clinical apparatus for inducing enhanced drug delivery via ultrasonic treatment.

While this patent describes a method for the use of low frequency ultrasound for particular drug delivery to enhance transdermal drug delivery, the method requires the use in a clinical ultrasonic delivery setting. Moreover, the time for delivery for measurable amounts using these methods range from 24 hours to 10 minutes. In this method, the use of ultrasound-transdermal drug delivery treatment is actually less desirable from a patient administration standpoint than a simple injection. This method is undesirable because of the need for the patient to visit the clinical setting and to remain on a treatment table while the ultrasound treatment is used to deliver the drug. This method causes damage to skin because the same area of the skin is treated continuously.

Ultrasound has also been used to enhance permeability of the skin and synthetic membranes to drugs and other molecules. Ultrasound has been defined as mechanical pressure waves with frequencies above 20 kHz, H. Lutz et al., Manual of Ultrasound 3-12 (1984). Ultrasound is generated by vibrating a piezoelectric crystal or other electromechanical element by passing an alternating current through the material, R. Brucks et al., 6 Pharm. Res. 697 (1989). The use of ultrasound to increase the permeability of the skin to drug molecules has been termed sonophoresis or phonophoresis.

U.S. Pat. Nos. 4,309,989 to Fahim describes topical application of medications in a coupling agent for the treatment of Herpes virus infections and demidox mite infestations. The medications are massaged into the affected area by ultrasound to cause the medication to penetrate the skin. U.S. Pat. No. 4,372,296 to Fahim similarly describes topical application of zinc sulfate and ascorbic acid in a coupling agent for treatment of acne.

U.S. Pat. No. 4,767,402 to Kost et al. discloses a method for enhancing and controlling infusion of molecules having a low rate of permeability through skin using ultrasound in the frequency range of between 20 kHz and 10 MHz, and in the intensity range of between 0 and 3 W/cm.sub.2. The molecules are either incorporated in a coupling agent or, alternatively, applied through a transdermal patch. Kost et al. further teach that the parameters of time, frequency, and power can be optimized to suit individual situations and differences in permeability of various molecules and of various skins. Transbuccal drug delivery with ultrasound has also been disclosed, U.S. Pat. No. 4,948,587 to Kost et al.

U.S. Pat. No. 5,115,805 to Bommannan et al. discloses the use of specific frequencies (i.e. >10 MHz) of ultrasound to enhance the rate of permeation of drugs through human skin into the body. Frequencies above 10 MHz gave improved penetration of the skin above that described earlier. It is alleged that chemical penetration enhancers and/or iontophoresis can also be used in connection with the ultrasound treatment to enhance delivery of drugs through the skin into the body.

U.S. Pat. No. 5,016,615 to Driller et al. involves local application of a medication by implanting a drug-containing receptacle adjacent to a body tissue to be treated and then applying ultrasound to drive the drug out of the receptacle and into the body tissue. This method has the disadvantage of requiring surgical implantation of the drug receptacle and a noninvasive technique is preferred. U.S. Pat. No. 4,821,740 to Tachibana et al. discloses a kit for providing external medicines that includes a drug-containing layer and an ultrasonic oscillator for releasing the drugs for uptake through the surface of the skin. U.S. Pat. No. 5,007,438 to Tachibana et al. describes an application kit in which a layer of medication and an ultrasound transducer are disposed within an enclosure. The transducer may be battery powered. Ultrasound causes the medication to move from the device to the skin and then the ultrasound energy can be varied to control the rate of administration through the skin.

Other references teaching the use of ultrasound to deliver drugs through the skin include D. Bommannan et al., 9 Pharmaceutical Res. 559 (1992); D. Bommannan et al., 9 Pharmaceutical Res. 1043 (1992); K. Tachibana, 9 Pharmaceutical Res. 952 (1992); P. Tyle & P. Agrawala, 6 Pharmaceutical Res. 355 (1989); H. Benson et al., 8 Pharmaceutical Res. 1991); D. Levy et al., 83 J. Clin. Invest. 2074 (1989).

Other methods of increasing the permeability of skin to drugs have been described, such as ultrasound or iontophoresis. Iontophoresis involves the application of an external electric field and topical delivery of an ionized form of drug or a unionized drug carried with the water flux associated with ion transport (electro-osmosis). While permeation enhancement with iontophoresis has been effective, control of drug delivery and irreversible skin damage are problems associated with the technique.

Thus, while the use of ultrasound for drug delivery is known, results have been largely disappointing in that enhancement of permeability has been relatively low. There is no consensus on the efficacy of ultrasound for increasing drug flux across the skin. While some studies report the success of sonophoresis, J. Davick et al., 68 Phys. Ther. 1672 (1988); J. Griffin et al., 47 Phys. Ther. 594 (1967); J. Griffin & J. Touchstone, 42 Am. J. Phys. Med. 77 (1963); J. Griffin et al., 44 Am. J. Phys. Med. 20 (1965); D. Levy et al., 83 J. Clin. Invest. 2074); D. Bommannan et al., 9 Pharm. Res. 559 (1992), others have obtained negative results, H. Benson et al., 69 Phys. Ther. 113 (1988); J. McElnay et al., 20 Br. J, Clin. Pharmacol. 4221 (1985); H. Pratzel et al., 13 J. Rheumatol. 1122 (1986). Systems in which rodent skin were employed showed the most promising results, whereas systems in which human skin was employed have generally shown disappointing results. It is well known to those skilled in the art, that rodent skin is much more permeable than human skin, and consequently the above results do not teach one skilled in the art how to effectively utilize sonophoresis as applied to transdermal delivery and/or monitoring through human skin.

Applicant suggests that another use for medically directed ultrasound is to induce the pancreas to release insulin in response to the ultrasonic signal, with or without the addition of medication. The parameters of ultrasound can be changed to create a resonance effect within the patient's body, essentially gently massaging the pancreas over time, and thereby inducing the pancreas to release insulin on demand. The control of the sonic frequency, intensity, and time of exposure is theorized by Applicant to create an effect for the non-invasive inducement of the pancreas to affect the release of insulin on demand. All three of these parameters may be modulated simultaneously in a complex fashion to increase the effect or efficiency of the ultrasound as it relates to enhancing the biomolecular flux rate of insulin from the patient's pancreas.

Applicant has discovered that miniature transducers, similar in design to those espoused in U.S. Pat. Nos. 5,729,077, 5,276,657 and 4,999,819 by Newnham, can be employed to direct a focused ultrasonic transmission through the body, aimed at a particular organ in the body, for the purpose of creating a mild harmonic effect internally within the patient's body. Applicant further theorizes that the focused ultrasonic transmission can be used to induce the organ to release bioactive compounds, which will aid in the fight against disease. One such application would be the inducement of the pancreas to release insulin on demand, in response to the ultrasonic transmission. Other applications of this invention could include the release of antibodies, endorphins or enzymes from other organs, tissue or even the skeletal structure to fight other ailments or disease.

### SUMMARY OF THE PRESENT INVENTION

Accordingly, the purpose of this invention is to provide a device for enhancing or increasing the release of insulin in a diabetic patient by the use of ultrasound, wherein the device is self powered, portable or worn by the patient, and programmable. The present invention is a wearable or otherwise portable, programmable, sonic applicator which is placed directly in contact with the skin, and ideally located directly above the pancreas of a diabetic patient, for the purpose of both enhancing and controlling the release of insulin in a Type-2 diabetic patient, with or without the use of accompanying medications. The device is placed directly on the patient's skin, and held in place by adhesive strips or body affixing straps. The sonic applicator, when activated by its internal timing circuitry, generates an ultrasonic vibration or ultrasonic transmission through the patient's body. While the exact mechanism by which ultrasound works to increase the release of insulin is not fully understood, the acoustic energy of the ultrasonic waves and vibration effects induced within the patient's pancreas is believed to increase the release of insulin emanating from the pancreas into the patient's bloodstream.

The sonic applicator is provided with control circuitry for regulating the timing, frequency, and intensity of the acoustic energy, which is applied to the patient's skin. The control circuitry can be set to apply a continuous ultrasound or vibratory mode whereupon the ultrasonic effect can be administered continuously. Alternatively, the control circuitry of the device can be set to pulse in various on-off cycles over the course of the treatment time. The duration of the cycles and their spacing over the course of a day can be programmed to produce a pulsed dosing of the released insulin to the patient.

The sonic applicator of this invention is wearable and therefore portable and may be attached to the patient by way of adhesive strips or a straps. The sonic applicator contains its own battery power supply. When power runs low, the batteries may either be replaced or recharged if rechargeable batteries are used. The combination of an ultrasonic applicator, worn by the patent provides several drug delivery advantages:

1) The sonic applicator can provide a non-invasive means of insulin delivery, but one, which relies upon inducing the patient's own pancreas to release the needed insulin dose on demand.

2) The sonic applicator can induce the patient's own pancreas to release the needed insulin dose on demand, with or without the use of accompanying medication. As such it reduces the need to rely upon medication, and for the patient to avoid the complications of the long-term use of such chemical formulations in the treatment of diabetes.

3) The system can be programmed to provide steady drug delivery or pulsed timed delivery at certain medication quantities, providing more flexibility and control over particular patients dosing needs. Conventional drug delivery systems are steady-state release devices providing a one size fits all regimen, which is not suited for all patent medication regimes.

Accordingly the primary object of the invention of the invention is a portable, programmable device using ultrasound to induce and controlling the release of internally produced insulin from a diabetic patient's pancreas.

Another object of the invention is a method for the non-invasive delivery of insulin through the use of ultrasound administered through the skin and directed to the pancreas of a diabetic patient, where the sonic therapy is administered solely, and without the use of any medication for the treatment of diabetes.

Another object of the invention is a method for the non-invasive delivery of insulin through the use of ultrasound administered through the skin and directed to the pancreas of a diabetic patient, where the sonic therapy is administered in conjunction with the use of appropriate medications for the treatment of diabetes.

These and other objects of the inventions can be accomplished by applying various ultrasound frequencies, intensities, amplitudes and/or phase modulations to control the magnitude of the transdermal flux to achieve a therapeutic release of insulin from a diabetic pancreas.

One aspect of the programmability and flux control is the ability to optimize therapeutic delivery for an individual patient (such examples may include patients that are at different stages of the disease, elderly patients, young, juvenile, or according to gender).

Another aspect is to optimize insulin release delivery for in conjunction with an appropriate chemotherapy regimen. The molecular structure of each biologically active molecule is different and responds differently to ultrasound. Control of the frequency, intensity, concentration, timing of delivery, and associated drug regimen can optimize effect of each drug type used with sonic therapy in diabetes treatment.

The preferred aspect of the invention is the use of the device for insulin therapy through inducing the delivery of insulin from a solicited pancreas. Applicant however recognizes that this same technology may be used to induce the release of other life saving compositions from other organs and as such does not wish to limit this invention solely to the treatment of diabetes. Moreover a change in pH or enzymes in the blood or liver or other organs in response to sonication may lead to other treatments of various ailments and diseases, by allowing targeting of the activity of the organ system or the blood with ultrasound therapy.

Another embodiment of the invention is the use of phase modulation, alternating waveforms and frequency modulation to achieve organ excitation and activation.

Another aspect of the invention is the combination of ultrasound with iontophoresis, electroporation, depilatories, or use with medication such as diabetic drugs to facilitate the release of insulin on demand from a diabetic's own pancreas or endocrine system.

Additional objects, advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 is an artist's depiction of the invention as it is placed on the abdomen of a patient.

FIG. 2 is a depiction of the control apparatus for inducing an ultrasonic signal through the skin of a patient, for the treatment of diabetes.

FIG. 3 depicts the use of an alternating waveform, a conversion from sawtooth to square wave, as generated by the frequency driver of this invention.

FIG. 4 illustrates the design of a transducer device suitable for use in this invention, utilizing a cymbal type design.

FIG. 5 is a photograph of the cymbal type transducer element.

FIG. 6 illustrates a design for the use of multiple transducers arranged in an array configuration.

FIG. 7 is a graph illustrating the normal glucose profile of a 350-gram test rat.

FIG. 8 is a graph illustrating the normal glucose profile of a 350-gram test rat exposed to ultrasound on a continuous basis for 30 minutes.

### DESCRIPTION OF THE PREFERRED EMBODIMENTS

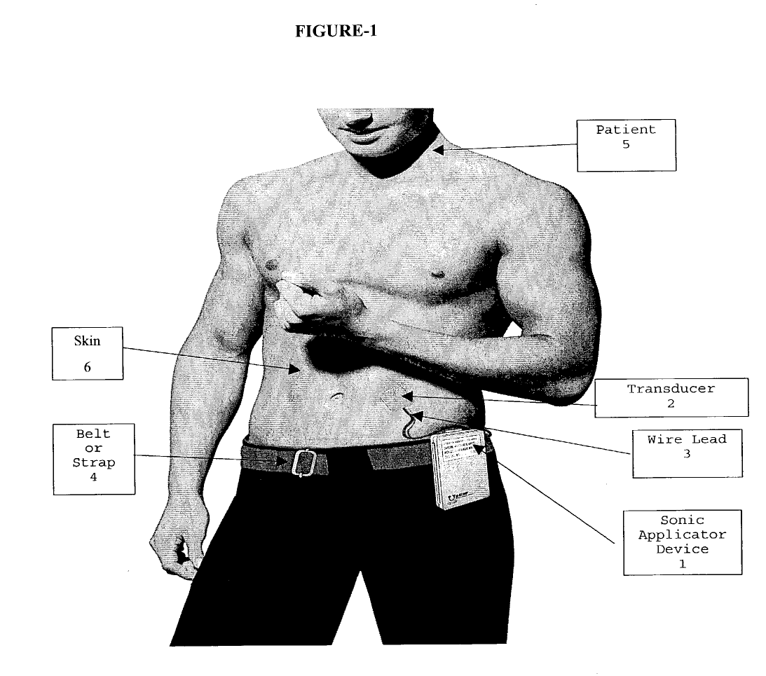

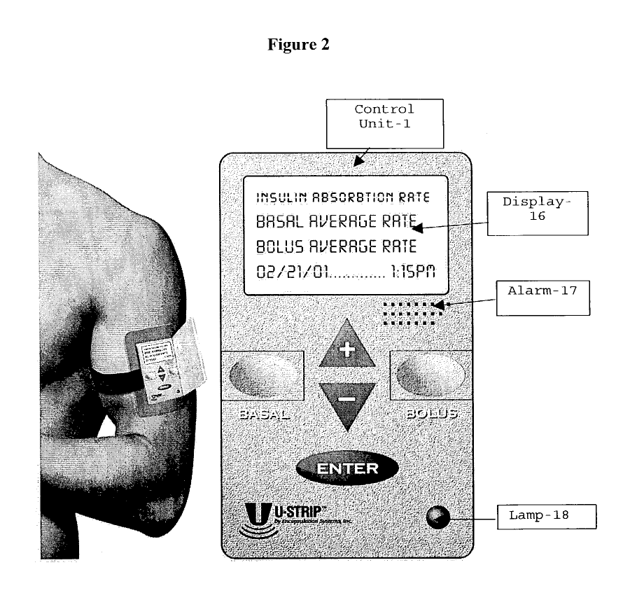

FIG. 1 illustrates the present invention comprising a sonic applicator device (1), which ideally is worn on the belt (4) of a patient. Alternatively the sonic applicator device (1) may be affixed to the patient by means of a strap and may in fact be located anywhere on the body where it is convenient to the patient to control the function of the device. An alternative position is shown in FIG. 2, mounted upon the arm. FIG. 1 illustrates that the preferred placement however, on the waist of the patient for treatment through the abdomen. The device is intended to go with the patient, to be wearable by the patient, containing rechargeable batteries to provide treatment mobility. This insures patient compliance with a drug regimen, because there is little for the patient to do but keep the system in place.

A transducer device (2) is affixed to the skin surface (6) of the patient (5) ideally over the target organ, which in the case of diabetes treatment may be the pancreas or the liver.

Power for the sonic applicator (1) is provided by power cells (not shown), which are ideally rechargeable, and may be located within the strap (4) itself.

Alternatively, the power supply may be contained within the sonic applicator device (1) itself or provided by an external source. To be totally portable, the unit contains a battery system within it, which may be replaceable and ideally is rechargeable. Alternately, an external battery pack may be worn and connected to the applicator. Applicant envisions the preferred system to have batteries, which are stored within the strap (4) of the device.

A single transducer or an array of ultrasonic transducers, which is controlled by a frequency generator and sonic driver circuit within the sonic applicator device (1), generates the sonic transmission. The circuitry controls the settings of the device and the activation sequencing.

A wire lead (3) connects the ultrasonic transducer assembly (2) to the sonic applicator device. The ultrasonic transducer assembly (2) may be any appropriate device suitable for the transmission of sonic or ultrasonic transmissions through the patient's body.

A coupling agent compound, such as a hydrogel, including non-water soluble agents such as silica gel may be used under the transducers to help maintain the contact with the skin and maintain the ultrasonic signal integrity.

Design of Transducer Element

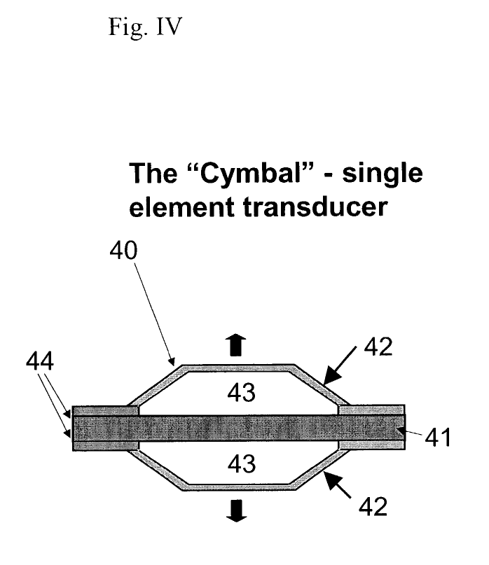

FIG. 4 illustrates the design of a cymbal type of ultrasonic transducer (40), which is the preferred embodiment of the transducer element of this invention. From FIG. 4 it can be seen that a cymbal transducer (40) is based upon a piezoelectric disc (41) such as PZT4 (Piezokinetics Corp. Bellefonte, Pa.), connected to two metal caps (42) composed of titanium foil preferably. FIG. 4 illustrates that there is a hollow air space (43) between the piezoelectric disc (41) and the end caps (42). The end caps (42) are connected to the piezoelectric disc (41) by a non-electrically conductive adhesive (44) to form a bonded layered construction to the transducer (40). The cymbal transducer offers a thin, compact structure more suited for a portable ultrasonic drug delivery apparatus. Additionally this transducer offers greater efficiency for the conversion of electric power to acoustically radiated power. Applicant chose this design of a transducer also because of its potential to be battery powered.

The use of low frequency ultrasound, ideally from 20-100 kHz, which uses alternating waveform (from sawtooth to square wave), with cymbal type transducers, which enable battery power ultrasonic transmission. A transducer array to avoid over exerting a single skin transport site and providing versatility in ultrasonic frequency and intensity ranges per transducer element.

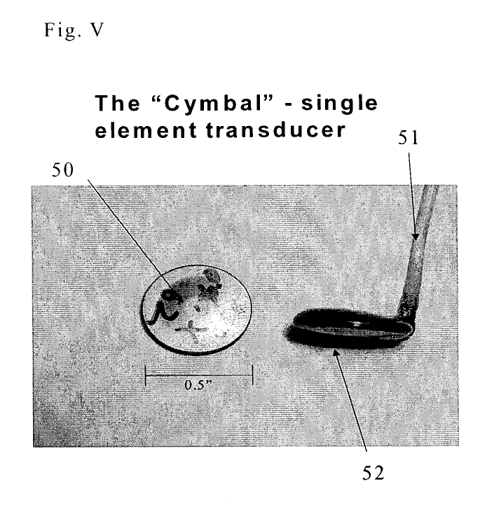

FIG. 5 illustrates that the cymbal transducer enables a compact and minute size to the transducer element of the invention. The sizing of the transducers was obtained at just 0.5″ inches diameter. The small size transducer was necessary in the invention to enable the transducers to fit within the dimensions of the Transdermal Patch. In addition the small size enabled a lower weight potential for the transducers, again aiding in the portability of the invention. The transducer element (50) is a cymbal type construction attached to a power cable (51). The transducer element (50) is coated in a polymer housing (52), ideally composed of uralite resin, which is used to avoid short circuits between the two metallic caps (42) (FIG. 4) and provides acoustic coupling for the sonic transmission.

The cymbal type transducer design offers several key advantages over the prior art:

Compact structure, with small surface area.

High acoustic pressure and high acoustic intensity at low resonance frequency.

High efficiency, making the system require less driving power.

The use of low resonance frequency is required to avoid a high cavitation threshold, i.e., the intensity required to generate air bubbles within the stratum corneum of the patient's skin tissue. The cavitation threshold is directly proportional to the frequency applied so the choice of a low resonance frequency of the transducer permits a lower acoustical pressure applied to the surface of the skin and transdermal drug delivery is affected.

Design of Transducer Array

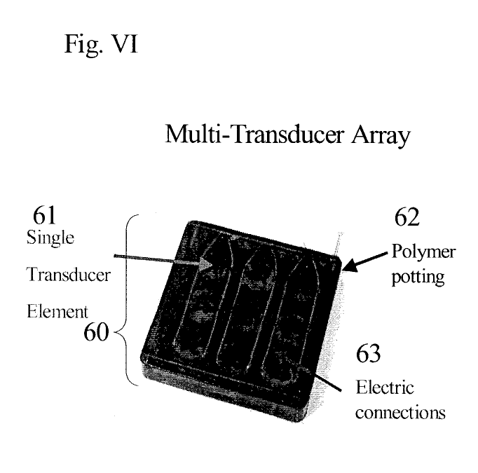

FIG. 6 shows an array (60) consisting of more than one-cymbal elements (61) arranged in an appropriate pattern onto a substructure or encased within a polymer housing (62). The cymbal elements (61) are connected in parallel by a series of electrical connections (63). Next, the array (60) is then sealed in polymer potting material (62) composed of uralite, preferably. The array enables a portable, battery powered ultrasonic transmission, with sufficient power to affect drug delivery via a transdermal patch.

In the preferred embodiment of this invention the transducers act in tandem, transmitting together. An alternate design could involve a transducer array whereby the activation of each element of the transducer array can be sequenced from transducer to transducer, possibly with different waveforms, frequency, amplitudes, and duty cycles between each transducer element. This has the affect of relieving the skin transport sites from continual ultrasonic stress and provides maximum variability in ultrasonic skin transport energy manipulation.

The transducer array as shown in FIG. 6 offers a means to spread out the drug pathway sites along the skin surface by providing ultrasonic transmission from the multiple transducer elements (61) of the array acting upon the skin. The transducer elements (61) may be activated simultaneously or sequentially to transmit ultrasound through the patch and through differing multiple sites on the skin surface. Additionally, the frequency, intensity and waveform may be altered at each transducer element (61) within the array (60). This variation has the effect of increased efficiency, enhanced power utilization and lengthening the life span of the battery of the portable system. Additionally, the alternating transducer elements (61) help to ensure that the skin is not overexposed to an excessive frequency of ultrasound.

An array of two or more transducers, especially the cymbal type, helps to push drugs through multiple skin transport sites. Moreover, the standard advantages of a transducer array reduce skin damage and improve the efficiency and transmitted acoustical intensity. By alternating the transducer activation sequence it is possible to avoid skin exertion and to assure greater longevity for the skin transport sites.

Using an array of transducers in a portable, wearable, ultrasonic drug delivery device, especially utilizing cymbal type transducers, provides higher power utilization efficiencies and helps to avoid the damaging effects of excessive cavitation upon the skin. The array makes possible long duration battery supplies providing sufficient power to enable the apparatus to function for several days between recharge or replacement cycles. The use of a rechargeable battery supply, ideally with batteries contained with the strap of the device, afford total mobility for the patient and a reliable power supply for the device over several months of recycled use.

FIG. 2 illustrates the control settings of the sonic applicator device (1) as designed for the control of insulin therapy via ultrasound. These controls permit the device to be programmed to deliver ultrasound, through the patient's body at prescribed intervals.

The KEYPAD (10) provides control functions, which can be set by either the patient or the medical professional. The current embodiment of the sonic applicator unit (1) has a keypad (10) providing several functions:

The ENTER key (15) is the on-off control button. When activated, it sends stored battery power to the other control elements and to the sonic applicator element. It also enables the operator to enter functions and commands displayed on the DISPLAY (16).

The diabetic patient needs a basal delivery schedule for the release of insulin. The BASAL key 1 (1) enables the operator to set the device to activate for a certain time interval to enable the device to transmit an ultrasonic signal, which will excite the patient's pancreas organ below the skins surface, and timed to release a certain quantity of insulin over time.

The diabetic patient may also need a bolus delivery schedule for the release of insulin, particularly at mealtime or during heightened physical activity. The BOLUS key (12) enables the operator to set the device to activate for a certain time interval to enable the device to transmit an ultrasonic signal, which will excite the patient's pancreas organ below the skins surface, and timed to release a certain quantity of insulin, generally in a short period of time.

Control (+), (13), enables the operator to scroll upward to the desired selection, based upon the time interval desired or the corresponding dose desired to be released upon ultrasonic excitation.

Control (−), (14), enables the operator to scroll downward to the desired selection, based upon the time interval desired or the corresponding dose desired to be released upon ultrasonic excitation.

Once a setting has been obtained, it is entered into the electronic logic circuit of the device through the enter key (15). The Display (16) illustrates the choice and the status of the device.

If the device encounters a confusing entry, or its self-diagnostic circuit illustrates a problem an audible alarm is sounded at the speaker (17) and a red indicator light activates (18). The Display (16) then becomes a message center to identify the problem and recommends a course of action.

The preferred embodiment of the sonic applicator device maintains a memory of all settings, self-diagnostic reports and activation sequences. This information can be downloaded via a modem to the medical professional that can then access the dosage history of the patient. With this data the medical professional can retune the device to meet the insulin therapy needs of a particular patient.

The device can be set by the medical professional, or used by the patient himself or herself in an insulin therapy program. Alternatively the device could receive signals from a glucose sensor and activate to excite the pancreas as needed to maintain a proper insulin therapy level. As such, this closed-loop system would function as an artificial pancreas.

The controls of the device activate a sonic driver circuit within the device, said sonic driver circuit being an oscillation circuit capable of providing the proper ultrasonic frequency and intensity level to the transducers to emanate the proper sonic transmission. The frequency and intensity generator setting is ideally preset for a specific frequency and intensity of acoustic energy delivery. Control settings establish the periodicity of ultrasonic application. Using this setting will insure that there are, for example, one hundred ultrasonic pulses per second in duration.

The controls can establish a steady delivery mode, which is continuous and lasts for several seconds when activated by the timed setting established by the various entered settings. For example, a single continuous delivery ultrasonic transmission lasting, for example, 10 seconds in duration of the frequency and intensity established by the timing circuit. Additionally, the device can be made to activate the sonic applicator function at pre-set times during the day, for specific treatment periods.

In certain cases, where the patient is required to participate in the management of his or her disease, the controls could be set to be patient operated. The treatment of diabetes often requires patients to self regulate their dosage of insulin and to administer booster doses before a meal. In such instances the controls would be accessible by the patient directly.

Applicant notes that most diabetic patients will need up to 36 units of insulin per day. Each unit is approximately 40 micrograms of active insulin. A typical Type-I diabetic patient, weight at 154 lbs., would utilize 1,440 micrograms per day or 1.4 milligrams. For Type-II diabetic patients the delivery needs may be varied according to their body's ability to produce insulin. To provide a basal delivery schedule suitable for diabetic patients there are several schedules, which can be entered into the timing circuit of the device.

| | | | Basal Rate Per |
|---|---|---|---|
| | | Basal Rate Per | Hour (hr) in |
| | | Hour (hr) in Units | Micrograms of |
| Setting Option | No. of Units/day | of Insulin | Insulin |
| 1 | 36 | 1.5 | 60 |
| 2 | 24 | 1.0 | 40 |
| 3 | 18 | 0.75 | 30 |
| 4 | 12 | 0.5 | 20 |

The keypad of the device would be programmed to deliver the proper ultrasonic dose across the skin, via the timing circuit, to induce the pancreas to release the proper quantity of insulin on demand.

Applicant realizes that the glucose levels of diabetic patients may vary from time to time and that a bolus amount of insulin may be required before a meal. The device of this invention can be designed to enable the diabetic patient to provide a bolus or booster quantity on demand prior to a meal. FIG. 2 shows the control panel of the device, which can be altered to meet the delivery regimen and controls for particular drug delivery protocols. The delivery of a bolus could be operated from control in the illustrated design, but alternative designs to the control function are possible. Applicant is aware of glucose monitors, who are coming upon the market, which claim to be non-invasive, and continuous. Such systems could be applied to the device to provide sensor data so that the unit provides an on demand quantity of drug in response to data coming from such a glucose monitor.

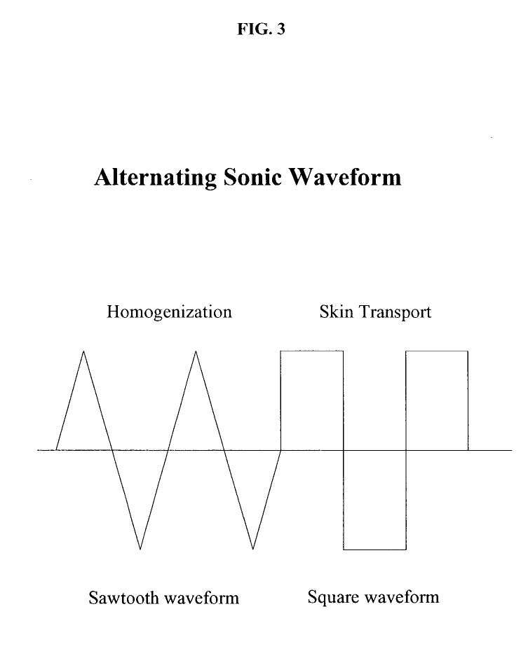

Applicant has also discovered that through the use of alternating waveforms the amount of energy transmitted to the surface of the skin could be minimized while also providing a pressure wave effect which transverses the skin into the interior structure of the body. Referring to FIG. 3 the preferred embodiment employs a waveform, which alternates from sawtooth to square wave. The amplitude of and intensity of the wave shaping is theorized to provide a harmonic resonance effect within the targeted organ. Applicant theorizes that the short, peaked portion of the ultrasonic waveform in a sawtooth shape helps avoid destructive frequencies and cavitation to the skin and to the underlying tissue. Upon conversion to the square waveform the ultrasonic transmission acts to massage the tissue or organ structures. In the case of the pancreas, applicant theorizes the harmonic effect causes a release of insulin and c-peptide levels within the organ directly into the bloodstream at the vascular network.

Acoustic energy waves, generated by the sonic applicator device (1), emanate from the transducer assembly (2) on the surface of the skin and transverse through the patient's tissue.

From there the ultrasonic transmission acts as a carrier wave, traveling through the skin layers, through the musculature of the patient and into the target organ. There the carrier wave is theorized to induce the organ to release enzymes, endorphins, or other biofunctional compounds within the organ. In the case of diabetes, the insulin released in response to the ultrasound transmission would travel directly into the blood stream.

The system would utilize ultrasound to enhance the ability of the pancreas to release insulin through the following operating modes:

1. Ultrasonic transmission activates or excites the pancreas into releasing insulin, without the use of accompanying medications.

2. Ultrasonic transmission activates or excites the pancreas into releasing insulin, with the use of accompanying medications, aimed at making the body more capable of utilizing the insulin.

3. Ultrasonic transmission activates or excites the medication to increase its effectiveness in insulin therapy or diabetes treatment.

As noted above, no one yet fully understands why the ultrasonic energy affects delivery of the drug molecules through the skin, but the effect has been well documented. Applicant has theorized that drug delivery can be affected via another means, inducing the organ to release biofunctional compounds on demand via the use of ultrasonic transmissions focused upon a particular organ within the body.

### Experiments

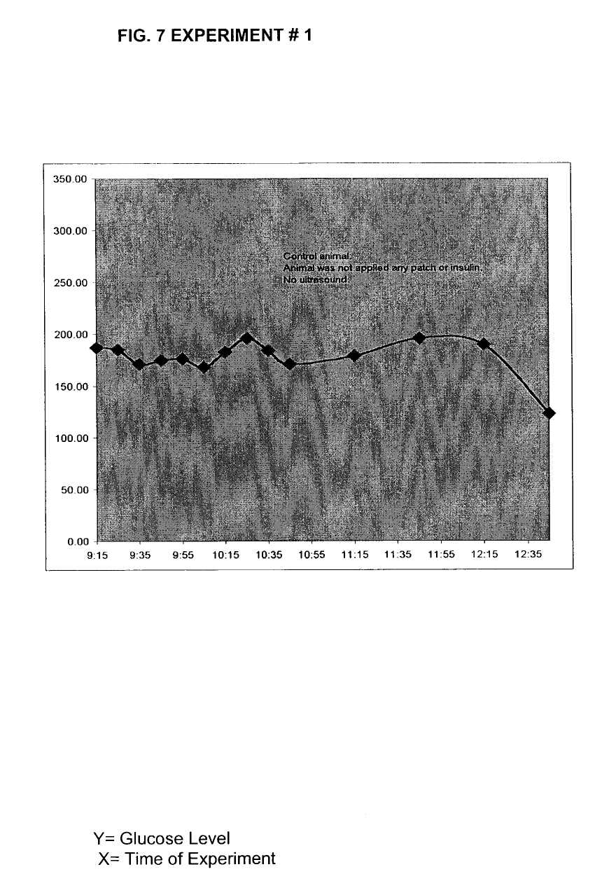

### Experiment #1: Depicted in the Graph of Fig. FIG. 7

A 350-gram test rat was anesticized and allowed to sit for over 30 minutes. Blood samples were taken intravenously from the rat every 10 minutes and those samples were tested by a glucometer device, known as a Model YSI 2300 Stat Plus Device, manufactured by YSI Inc., for glucose level. FIG. 7 is a graph illustrating the normal glucose profile of a normal test rat. The Y-axis is the glucose level measured in mg/dl of blood. This test provides a baseline glucose level for the normal rat.

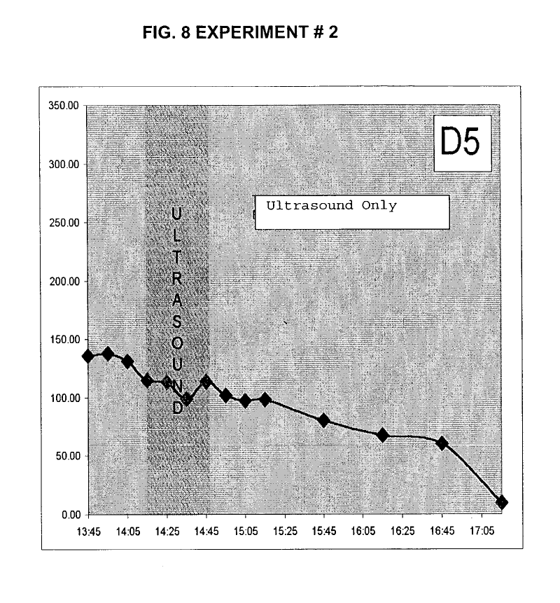

### Experiment #2: Depicted in the Graph of Fig. FIG. 8.

A 350-gram test rat was anesticized and allowed to sit for over 30 minutes, at which time the animal was subjected to ultrasound for another 30 minutes. The ultrasound level was 17 kHz frequency at 125 mW/sq. cm intensity.

Blood samples were taken intravenously from the rat every 10 minutes and those samples were tested by a glucometer device, known as a Model YSI 2300 Stat Plus Device, manufactured by YSI Inc., for glucose level.

FIG. 8 is a graph illustrating the glucose profile of a 350-gram test rat exposed to ultrasound on a continuous basis for 30 minutes. The Y-axis is the glucose level measured in mg/dl of blood. In this experiment it can clearly be seen that ultrasound alone can be utilized to effect a drop in the glucose level of a living animal. It is theorized that the glucose drop was effected by a release of insulin from the pancreas of the test animal.

### SUMMARY

To facilitate the release of insulin the invention allows several key advantages over the previous art:

1. The invention as described herein provides a portable device, which can be adjusted to deliver the basal and bolus delivery of insulin at various timing intervals to match the delivery needs of particular patients, through the use of ultrasonic transmissions focused upon the pancreas, as a means of inducing the pancreas to release insulin on demand by the device.

2. The portability is provided by a wearable device and not just a device, as envisioned in the previous art, which is carried by the patient and administered on a schedule to be remembered and adhered to by the patient.

3. The invention provides a system for the inducement of insulin—with or without the use of accompanying chemotherapy or medications.

4. The invention provides a means of both basal and bolus insulin delivery via a programmed system, timed to administer the particular dosage of insulin needed for that particular patient's drug delivery regimen through ultrasonic excitation of the patient's pancreas by ultrasound.

5. Applicant notes that transmissions in both the sonic and ultrasonic ranges may prove effective in this invention.

6. The portable programmable sonic applicator of this invention will improve the quality of life for patients with diseases or conditions which require periodic administration of drugs by permitting the patients to continue the routines of their daily lives while providing appropriate medication. In addition, the cognitively impaired, elderly, and very young may receive medication with much less supervision.

Having described the invention in the above detail, those skilled in the art will recognize that there are a number of variations to the design and functionality for the device, but such variations of the design and functionality are intended to fall within the present disclosure.

I claim

1. A method for inducing the release of active biopharmaceutical compositions from various organs, glands or other bio-structures within the body of an individual, comprising applying ultrasound through a transducer device which is affixed to the exterior of the patient's body and worn by the patient during the day, said transducer device controlled by a portable, programmable ultrasonic regulator device, which itself is worn by the individual wherein said ultrasound is applied at an intensity and for a time period effective to enable movement of a therapeutic quantity of said active pharmaceutical composition from the target organ, glands or other bio-structures for the purpose of effecting regulated, and timed drug delivery of said of active biopharmaceutical composition(s) to the individual.

2. The method of claim 1, wherein said ultrasound has a frequency in the range of about 20 kHz to 10 MHz.

3. The method of claim 1, wherein said intensity of said ultrasound is in the range of about 0.01 W/cm.sup.2 to 5.0 W/cm.sup.2.

4. The method of claim 1, wherein the wearable, portable sonic device is affixed onto or connects to a transducer device, which provides the delivery of ultrasound, targeted to a specific organ, glands or other bio-structures of the individual.

5. The method of claim 1, wherein the ultrasound signal is composed of a waveform pattern which alternates, for example from sine wave to square wave or from sawtooth wave to square wave, as a means of injecting sonic energy while simultaneously minimizing the effect upon the skin or the internal body structures or organs of sonic cavitation.

6. The method of claim 1, wherein the ultrasound is applied continuously.

7. The method of claim 1, wherein the ultrasound is pulsed.

8. Apparatus as in claim 1, wherein transducer assembly is composed of a single cymbal type ultrasonic transducer.

9. An apparatus as claimed in claim 1, wherein cymbal type ultrasonic transducers are employed in transducer assembly array.

10. A means of transmitting ultrasound to a target organ within a patient's body, as claimed in claim 1, wherein the multiple transducer elements transmit ultrasound at identical frequencies and intensity levels to each other.

11. A means of transmitting ultrasound to a target organ within a patient's body, as claimed in claim 1, wherein the multiple transducer elements transmit ultrasound at differing frequencies and intensity levels to each other.

12. The assembly according to claim 1, wherein said transducer is a class V flextensional transducer “cymbal” type electrically connected with said ultrasonic waveform generator.

13. The assembly according to claim 1, wherein said transducer is an array consisting of several class V flextensional transducers, cymbal type elements electrically connected with said ultrasonic waveform generator.

14. The assembly according to claim 1, including a microprocessor means at least one basal timing sequence and at least one bolus timing sequence for effecting the inducement of an organ to release said biopharmaceutical compositions.

15. A method for inducing the release of insulin from within the pancreas of an individual, comprising applying ultrasound through a transducer device which is affixed to the exterior of the patient's body and worn by the patient during the day, said transducer device controlled by a portable, programmable ultrasonic regulator device, which itself is worn by the individual wherein said ultrasound is applied at an intensity and for a time period effective to enable movement of a therapeutic quantity of insulin from the pancreas for the purpose of effecting regulated, and timed drug delivery of said insulin to the individual.

16. The method of claim 15, wherein the ultrasound transmission is used to affect the release of insulin from the pancreas for the treatment of Type-1 and Type-2 diabetes.

17. A method for inducing the release of active biopharmaceutical compositions from various structures within the body of an individual, comprising applying ultrasound through a transducer device which is implanted within the patient's body and worn by the patient during the day, said transducer device controlled by a portable, programmable ultrasonic regulator device, which itself is either implanted or worn by the individual on the exterior of the body, wherein said ultrasound is applied at an intensity and for a time period effective to enable movement of a therapeutic quantity of said active pharmaceutical composition from the target organ, for the purpose of effecting regulated, and timed drug delivery of said of active biopharmaceutical composition(s) to the individual.
